(19) United States
(12) Patent Application Publication
(10) Pub. No.: US 2022/0305019 A1
(43) Pub. Date: Sep. 29, 2022

(54) PARAXANTHINE-BASED COMPOSITIONS FOR PROMOTING WEIGHT LOSS

(71) Applicant: Ingenious Ingredients, LP, Lewisville, TX (US)

(72) Inventors: Martin Purpura, Spring, TX (US); Ralf Jäger, Whitefish Bay, WI (US); Shawn Wells, Frisco, TX (US); Kylin Liao, Plano, TX (US)

(21) Appl. No.: 17/701,349

(22) Filed: Mar. 22, 2022

Related U.S. Application Data
(60) Provisional application No. 63/164,220, filed on Mar. 22, 2021.
(60) Provisional application No. 63/164,477, filed on Mar. 22, 2021.

(51) Int. Cl. A61K 31/522; A61K 36/82; A61K 31/205; A61K 31/165; A61K 31/475; A61K 36/38; A61K 36/258; A61K 31/145; A61K 31/352; A61K 31/045; A61K 38/17; A61K 31/593; A61K 31/122; A61K 33/06; A61K 36/81; A61K 31/4178; A61K 31/198; A61K 31/197; A61K 31/12; A61K 31/685; A61K 33/04; A61P 3/04

(52) U.S. Cl. CPC A61K 31/522; A61K 36/82; A61K 31/205; A61K 31/165; A61K 31/475; A61K 36/38; A61K 36/258; A61K 31/145; A61K 31/352; A61K 31/045; A61K 38/1767; A61K 31/593; A61K 31/122; A61K 33/06; A61K 36/81; A61K 31/4178; A61K 31/198; A61K 31/197; A61K 31/12; A61K 31/685; A61K 33/04; A61P 3/04

(57) ABSTRACT

Disclosed herein are methods for promoting weight loss in a subject by administering to the subject a composition comprising from about 2 mg to about 800 mg of paraxanthine. In certain aspects, paraxanthine is present in the composition in amount from about 20 mg to about 600 mg. In further aspects, paraxanthine is present in the composition in amount from about 50 mg to about 400 mg. According to certain embodiments, weight loss is promoted through inducing thermogenesis in the subject. In further embodiments, weight loss is promoted through enhancing lipolysis in the subject. According to certain implementations, administration of the composition to the subject decreases the respiratory quotient in the subject by at least about 10%.

### CROSS-REFERENCE TO RELATED APPLICATION(S)

This application claims priority to U.S. Provisional Application No. 63/164,220 filed Mar. 22, 2021 and entitled “PARAXANTHINE-BASED COMPOSITIONS FOR PROMOTING WEIGHT LOSS,” and U.S. Provisional Application No. 63/164,477 filed Mar. 22, 2021 and entitled “PARAXANTHINE-BASED COMPOSITIONS FOR PROMOTING SWAGGER,” each of which is hereby incorporated by reference in its entirety under 35 U.S.C. § 119(e).

### TECHNICAL FIELD

The disclosed technology relates generally to compositions, methods, and system for utilizing paraxanthine alone and in combination for use in supporting weight loss and/or improving body composition and/or fat loss through increased thermogenesis and/or suppressing the appetite and/or increased lipolysis. More particularly, the disclosure relates to paraxanthine and other compounds, whether produced synthetically or derived from natural sources, and use of these chemical compounds to provide physiological benefits, which may vary according to paraxanthine concentration and the presence of synergists and antagonists.

### BACKGROUND

Obesity is a significant problem in the Western world, with estimates of its prevalence ranging from 30% to 50% of the middle-aged population. The number of overweight (defined as a person with a body mass index (BMI) equal to or greater than 25 kg/m²) and obese (defined as a person with a BMI equal to or greater than 30 kg/m²) Americans has continued to increase since 1960, a trend that is not slowing down. Today, approximately 64.5% of adult Americans (about 199 million) are categorized as being overweight or obese. Obesity is becoming a growing concern as the number of people with obesity continues to increase and more is learned about the negative health effects of obesity. Obesity can lead to type 2 diabetes, heart disease, and some cancers. Each year, obesity causes at least 300,000 deaths in the U.S., and healthcare costs of American adults with obesity amount to more than $147 billion. There is a need in the art for compositions and methods to more effectively achieve weight loss.

### BRIEF SUMMARY

Disclosed herein are methods for promoting weight loss in a subject by administering to the subject a composition comprising from about 2 mg to about 800 mg of paraxanthine. In certain aspects, paraxanthine is present in the composition in amount from about 20 mg to about 600 mg. In further aspects, paraxanthine is present in the composition in amount from about 50 mg to about 400 mg.

According to certain embodiments, weight loss is promoted through inducing thermogenesis in the subject. In certain exemplary implementations, the composition further comprises one or more compounds selected from a list consisting of: caffeine, green tea, capsaicin, garcinia cambogia, yohimbine, catechols, epigallocatechin gallate (EGCG), catechins, and proanthocyanidins and octacosanol, and bitter orange.

In certain embodiments, weight loss is promoted through suppression of appetite in the subject and wherein administration of the composition to the subject suppresses appetite in the subject by at least about 30%.

In further embodiments, weight loss is promoted through enhancing lipolysis in the subject. According to certain implementations, administration of the composition to the subject decreases the respiratory quotient in the subject by at least about 10%.

In certain embodiments, resting energy expenditure in the subject is increased by at least about 15%. In certain implementations of these embodiments, the composition further comprises one or more compounds selected from a list consisting of caffeine, green tea extract, L-carnitine, Garcinia cambogia (hydroxycitric acid), capsaicin, ginseng, taurine, silk peptides and octacosanol.

According to certain embodiments, the method further includes restricting calorie intake of the subject. In exemplary implementations of these embodiments, the amount of weight loss in the subject is greater than that for a subject with an equivalent calorie restriction that has not been provided the composition and wherein the ratio of fat loss to muscle loss in the subject is greater than that for a subject with an equivalent calorie restriction that has not been provided the composition.

According to certain embodiments, the subject is not administered caffeine. In exemplary implementations, the subject abstains from consumption of caffeine.

Further disclosed herein is a method for suppressing appetite in a subject comprising: administering to the subject a composition comprising from about 2 mg to about 800 mg of paraxanthine. According to certain implementations, administration of the composition decreases appetite in the subject by at least about 30%. In certain implementations, the subject is not administered caffeine.

Further disclosed herein is a method for increasing swagger in a subject by providing the subject with a composition comprising about 2 mg to about 800 mg of paraxanthine. In certain embodiments, paraxanthine is present in the composition in amount from about 20 mg to about 600 mg. In further implementations, paraxanthine is present in the composition in amount from about 50 mg to about 400 mg.

According to certain implementations, the composition further comprises one or more compounds selected from the list consisting of: omega-3 fatty acids, vitamin D, vitamin B, protein, selenium, fast digestive carbohydrates like sugar, vitamin K, calcium, vitamin A, ashwagandha (Withania somnifera), Acetylcholine, Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna, L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, curcuminoids, caffeine, creatine, and Solanum asperum. In exemplary implementations, administration of the composition to the subject increases swagger in the subject by at least about 20%.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g. a subject. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more body weight disorders prior to the administering step. In some aspects of the disclosed method, the subject has been diagnosed with a need for increasing weight loss prior to the administering step.

The term “overweight” is defined as the condition wherein the individual has a BMI greater than or 25 kg/m2 and less than 30 kg/m2. The terms “overweight” and “pre-obese” are used interchangeably.

As used herein, the term “obesity” is defined as the condition wherein the individual has a BMI equal to or greater than 30 kg/m2. According to a WHO definition the term obesity may be categorized as follows: the term “class I obesity” is the condition wherein the BMI is equal to or greater than 30 kg/m2 but lower than 35 kg/m2; the term “class II obesity” is the condition wherein the BMI is equal to or greater than 35 kg/m2 but lower than 40 kg/m2; the term “class III obesity” is the condition wherein the BMI is equal to or greater than 40 kg/m2.

As used herein, the terms “manage,” “managing,” and “management” encompass preventing, delaying, or reducing the severity of a recurrence of an adipose associated body composition or body weight disorder, such as obesity, lypodystrophy, diabetes or metabolic syndrome, fibrosis and cancer in a patient who has already suffered from such a disease, disorder or condition. The terms encompass modulating the threshold, development, and/or duration of the adipose associated body composition or body weight disorder, such as obesity, lypodystrophy, diabetes or metabolic syndrome, fibrosis and cancer or changing how a patient responds to the adipose associated body composition or body weight disorder.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause unacceptable adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with obesity” means having been subjected to a physical examination by a person of skill, for example, a physician, and found to have a condition that can be diagnosed or treated by a compound or composition that can reduce body mass. As a further example, “diagnosed with a need for weight loss” refers to having been subjected to a physical examination by a person of skill, for example, a physician, and found to have a condition characterized by excess of body fat or other disease wherein decreasing body fat would be beneficial to the subject. Such a diagnosis can be in reference to a disorder, such as obesity, metabolic syndrome, and the like, as discussed herein.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is substantially free of particles would either completely lack particles, or so nearly completely lack particles that the effect would be the same as if it completely lacked particles. In other words, a composition that is substantially free of an ingredient or element may still actually contain such item as long as there is no measurable effect thereof.

As used herein, the term “synergistic effect” or grammatical variations thereof means and includes a cooperative action encountered in a combination of two or more active compounds in which the combined activity of the two or more active compounds exceeds the sum of the activity of each active compound alone. The term “synergistically effective amount,” as used herein, means and includes an amount of two or more active compounds that provides a synergistic effect defined above.

This disclosure relates to the use chemical compositions comprising paraxanthine. In certain embodiments, the composition comprises other chemicals, including paraxanthine congeners or analogs, to provide a plurality of desirable effects. Paraxanthine analogs may include, but are not limited to, caffeine, methyl caffeine, theobromine, theophylline, liberine and methylliberine, and their variants. Other suitable actives may include one or more weight loss supporting compounds such, capsaicin, Aframomum Melegueta Extract (Grains of Paradise), Green Tea Extract, Yohimbine, Black Pepper Fruit Extract, Cayenne Pepper Fruit Extract, Fucoxanthin, Ketogenic Bioactives like MCTs, Omega-3s in combination with fat down-breaking digestive enzymes, Green Tea Extracts Camellia Sinensis, 7-Keto (3-acetyl-7-oxo-dehydroepiandrosterone, African mango (Irvingia gabonensis), Decaffeinated Green Coffee Bean Extract, Naringin, Hesperidin, Green coffee bean extract (Coffea arabica, Coffea canephora, Coffea robusta), Bitter Orange Extract (Citrus aurantium-amine p-synephrine), a Crocus sativus L extract (saffron stigma), Glucomannan, Chromium Picolinate, Conjugated linoleic acid, PHGG fiber, β-Glucans, Hoodia gordonii, Irvingia gabonensis, Forskolin, Raspberry Ketone, Chitosan, White Kidney Bean Extract, Garcinia cambogia, Ashwagandha extract, Kava Kava, L-Tyrosine, 5-HTP, N-Acetyl-Tyrosine, Melatonin, Magnolia bark, Valerian and hops, Rhodiola Rosea, Apocynum venetum, Saffron, Agmatine, Salvia Sclarea (Clary Sage), Vitamins B1/B6/B12, Rhodiola Rosea, CoQ10, Citrulline, Beet root powder, Gaia herbs, Guarana extract, Ginseng extract, D-Ribose, catechols, epigallocatechin gallate (EGCG), catechins, and proanthocyanidins and octacosanol and the like.

In certain embodiments, the disclosed compositions are nutraceutical compositions. Exemplary nutraceutical compositions of the present disclosure may be formulated or used as a stand alone composition, or as a nutritional or bioactive component in food, a functional food, a beverage, a bar, a food flavor, a medical food, a dietary supplement, or an herbal product. A medium generally accepted in the art includes all pharmaceutically or nutraceutically acceptable carriers, diluents or excipients therefor.

Paraxanthine exhibits a wide variety of effects depending on dosage. The presence of other ingredients may also modulate its effects. It may be used to improve lipolysis, thermogenesis, and/or decreased appetite. In one embodiment, paraxanthine may be used to promote weight loss by reducing appetite, act as an antioxidant and as an anti-inflammatory. Paraxanthine may be used transdermal to enhance one or more of these effects. In another embodiment, a dietary supplement comprising about 2 mg to about 800 mg paraxanthine, either natural through fermentation or synthetic, is provided. In another embodiment, a nutritional supplement to support weight loss and/or fat loss through lipolysis for improving the body composition is provided. It is therefore an object of the present disclosure to provide compositions including paraxanthine capable of imparting a plurality of positive effects on a subjects body composition. It is another object of the present disclosure to provide congeners, derivatives and iterations of paraxanthine and synthetic chemical equivalents of paraxanthine. It is another object of the present disclosure to provide agglomerated paraxanthine, paraxanthine salts, microencapsulated, liposomal or esterified paraxanthine. It is another object of the present disclosure to provide paraxanthine combined with glycerides, propylene glycol, polyethylene glycol (PEG), lauroyl macrogol, lauroyl macrogol derivatives and co-crystallization products of paraxanthine.

Further disclosed herein is a method for promoting weight loss and/or weight management in subject by providing the subject with a composition comprising about 2 mg to about 800 mg of paraxanthine. Although doses will vary from subject to subject, suitable daily doses are in the range of about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) per subject, administered in single or multiple doses In certain aspects, paraxanthine is present in the composition in amount from about 20 mg to about 600 mg. In further aspects, paraxanthine is present in the composition in amount from about 50 mg to about 400 mg.

According to certain embodiments, weight loss is promoted through inducing thermogenesis in the subject. According to exemplary implementations of these embodiments, the composition may also include one or more compounds selected from: caffeine, green tea, capsaicin, garcinia cambogia, yohimbine, catechols, EGCG, catechins, and proanthocyanidins and octacosanol and bitter orange.

In certain implementations, administration of the disclosed compositions decreases the respiratory quotient (RQ) of the subject, relative to the RQ of the subject when administered a placebo. The term “respiratory quotient” as used herein refers to the ratio of: carbon dioxide given off by a human and oxygen consumed by a human, particularly under known resting conditions upon receiving a placebo. In certain embodiments, the reduction in the subject's RQ following administration of the disclosed compositions is from about 5% to about 70%. In further embodiments, reduction of the subjects RQ is from about 10% to about 60%. In yet further embodiments, reduction of the subject's RQ is from about 20% to about 50%. In still further embodiments, reduction of the subject's RQ is at least about 30%.

According to further embodiments, weight loss is promoted through suppression of appetite in the subject. In exemplary implementations of these embodiments, the composition further may include one or more compounds selected from: fenugreek, glucomannan, Gymnema sylvestre, 5-HTP, Caralluma fimbriata, green tea extract, Conjugated linoleic acid, Garcinia cambogia, and Yerba mate.

According to still further embodiments, weight loss is promoted through enhancing lipolysis in the subject. In exemplary implementations of these embodiments, the composition further may include one or more compounds selected from caffeine, green tea extract, L-carnitine, Garcinia cambogia (hydroxycitric acid), capsaicin, ginseng, taurine, silk peptides, catechols, EGCG, catechins, and proanthocyanidins and octacosanol and octacosanol.

According to certain implementations, the disclosed method further comprises restricting calorie intake of the subject. In exemplary implementations, the amount of weight loss in the subject is greater than that for a subject with an equivalent calorie restriction that has not been provided the composition. According to further implementations, the ratio of fat loss to muscle loss in the subject the subject is greater than that for a subject with an equivalent calorie restriction that has not been provided the composition.

Further disclosed herein is a method for promoting fat loss in subject by providing subject with a composition comprising about 2 mg to about 800 mg of paraxanthine. Depending upon the subject to be treated and the route of administration, the compounds of the invention may be administered at varying doses. Although doses will vary from subject to subject, suitable daily doses are in the range of about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) per subject, administered in single or multiple doses. In certain aspects, paraxanthine is present in the composition in amount from about 20 mg to about 600 mg. In further aspects, paraxanthine is present in the composition in amount from about 50 mg to about 400 mg.

According to certain embodiments, fat loss is promoted through inducing thermogenesis in the subject. According to exemplary implementations of these embodiments, the composition may also include one or more compounds selected from: caffeine, green tea, capsaicin, garcinia cambogia, yohimbine, catechols, epigallocatechin gallate EGCG, catechins, and proanthocyanidins and octacosanol and bitter orange.

According to further embodiments, fat loss is promoted through suppression of appetite in the subject. In exemplary implementations of these embodiments, the composition further may include one or more compounds selected from: fenugreek, glucomannan, Gymnema sylvestre, 5-HTP, Caralluma fimbriata, green tea extract, Conjugated linoleic acid, Garcinia cambogia, and Yerba mate.

According to still further embodiments, fat loss is promoted through enhancing lipolysis in the subject. In exemplary implementations of these embodiments, the composition further may include one or more compounds selected from caffeine, green tea extract, L-carnitine, Garcinia cambogia (hydroxycitric acid), capsaicin, ginseng, taurine, silk peptides and octacosanol.

In certain embodiments, the disclosed composition, when administered to a subject, increases the subjects resting energy expenditure, relative to the subject's baseline level or following administration of a placebo. In certain embodiments, the increase in the subject's resting energy expenditure following administration of the disclosed compositions is from about 3% to about 30%. In further embodiments, increase in the subject's resting energy expenditure following administration of the disclosed compositions is from about 5% to about 25%. In yet further embodiments increase in the subject's resting energy expenditure following administration of the disclosed compositions is from about 8% to about 20%. In still further embodiments, increase in the subject's resting energy expenditure following administration of the disclosed compositions is from about 10%.

According to certain implementations, the disclosed method further comprises restricting calorie intake of the subject. In exemplary implementations, the amount of fat loss in the subject is greater than that for a subject with an equivalent calorie restriction that has not been provided the composition. According to further implementations, the ratio of fat loss to muscle loss in the subject the subject is greater than that for a subject with an equivalent calorie restriction that has not been provided the composition.

Further disclosed herein are methods for suppressing appetite in a subject by administering to the subject a composition comprising about 2 mg to about 800 mg of paraxanthine. In certain embodiments, administration of the composition to the subject reduces the subject's appetite by from 5% to about 70%. In further embodiments, reduction of the subject's appetite is from about 10% to about 60%. In yet further embodiments, reduction of the subject's appetite is from about 20% to about 50%. In still further embodiments, reduction of the subject's appetite is at least about 30%.

According to certain embodiments of the disclosed method, the composition is administered in a therapeutically effective amount. In further embodiments, the composition is administered in a prophylactically effective amount.

In certain aspects, disclosed herein are methods to promote weight loss through the administration of an effective amount of one or more compositions disclosed herein. According to certain aspects, administration of effective amounts of the disclosed compositions are used in treating diabetes mellitus; preventing, slowing progression of, delaying or treating of a condition or disorder selected from the group consisting of complications of diabetes mellitus; preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity, metabolic syndrome and gestational diabetes; or improving glycemic control and/or for reducing of fasting plasma glucose, of postprandial plasma glucose and/or of glycosylated hemoglobin HbA1c; or preventing, slowing, delaying or reversing progression from impaired glucose tolerance IGT), impaired fasting blood glucose (IFG), insulin resistance and/or from metabolic syndrome to type 2 diabetes mellitus; or preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischemia, arteriosclerosis, myocardial infarction, stroke and peripheral arterial occlusive disease; or reducing body weight and/or body fat or preventing an increase in body weight and/or body fat or facilitating a reduction in body weight and/or body fat; or preventing, slowing, delaying or treating diseases or conditions attributed to an abnormal accumulation of ectopic fat; or maintaining and/or improving the insulin sensitivity and/or for treating or preventing hyperinsulinemia and/or insulin resistance; preventing, slowing progression of, delaying, or treating new onset diabetes after transplantation (NODAT) and/or post-transplant metabolic syndrome (PTMS); preventing, delaying, or reducing NODAT and/or PTMS associated complications including micro- and macrovascular diseases and events, graft rejection, infection, and death; treating diabetes associated with cystic fibrosis treating hyperuricemia and hyperuricemia associated conditions; treating or prevention kidney stones; treating hyponatremia; in a patient in need thereof.

Another aspect encompasses a combination therapy to regulate fat storage, energy utilization, and/or weight loss in a subject. In an exemplary embodiment, a combination for increasing energy utilization, decreasing body fat or for promoting weight loss may include combining the methods and compositions disclosed with a procedure or therapy such as a pharmaceutical therapy, gastric bypass, duodenojejunal bypass, biliopancreatic diversion, vertical sleeve gastrectomy, adjustable gastric banding, vertical banded gastroplasty, intragastric balloon therapy, gastric plication, Magenstrasse and Mill, small bowel transposition, biliary diversion, brown adipose tissue modulation (e.g., controlled activation, enhanced differentiation, supplemental implantation, etc.), pharmaceutical administration, electrical stimulation of nerves that innervate at least a portion of the gastrointestinal tract, therapies impacting circadian rhythms, bile acid modulation, intestinal mucus production and metabolism, duodenal endoluminal barrier or similar manipulations of the gastrointestinal tract. For example, a composition dileucine can be administered to the subject prior to, concurrently with or after a gastric bypass or other gastrointestinal or bariatric procedure.

In certain aspects, administration of the disclosed compositions is effective at preventing reducing body weight and/or body fat or preventing an increase in body weight and/or body fat or facilitating a reduction in body weight and/or body fat; or preventing, slowing, delaying or treating diseases or conditions attributed to an abnormal accumulation of liver fat; or maintaining and/or improving the insulin sensitivity and/or for treating or preventing hyperinsulinemia and/or insulin resistance.

An advantage of using the invention may be the reduced likelihood that a person develops a tolerance to chemical compositions in accordance with the principles of the disclosure. That is, a person may not become desensitized to the effects induced.

In another embodiment, paraxanthine may be used as a topical agent for incorporation into body creams or lotions to produce a cream or lotion for lightening skin, firming skin, and/or improving skin elasticity. A paraxanthine topical agent may also be used to promote localized transdermal fat loss. Paraxanthine may also be used in a cream or lotion to promote localized enhanced metabolism and/or enhanced thermogenesis.

The administration of the disclosed compositions to a subject may include any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

According to certain embodiments, disclosed composition are administered to a subject in order to promote or enhance swagger in the subject. Swagger is a confident self-assured behavior that is a common characteristic of many successful people including business leaders, celebrities, and successful athletes. Many people who lack confidence or swagger feel impeded in personal or profession endeavors. Thus, there is a need in the art to identify alternative chemical compounds and mixtures thereof that may provide benefit to confidence and/or swagger. It is also desirable to provide chemical compounds and mixtures thereof that may be used to provide a variety of benefits, varying by concentration, thus requiring production of fewer materials.

Disclosed herein is disclosed herein is a method for increasing swagger in subject by providing the subject with a composition comprising from about 2 mg to about 800 mg of paraxanthine. In certain aspects, paraxanthine is present in the composition in amount from about 20 mg to about 600 mg. In further aspects, paraxanthine is present in the composition in amount from about 50 mg to about 400 mg.

In certain aspects, the composition further comprises one or more additional active ingredient to further enhance confidence. According to exemplary implementations, the one or more additional ingredient is selected from omega-3 fatty acids, vitamin D, vitamin B, protein, selenium, fast digestive carbohydrates like sugar, vitamin K, calcium, vitamin A, ashwagandha (Withania somnifera), Acetylcholine, Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, tetrahydrocurcumin, and Solanum asperum.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods.

As used herein, the term “functional food” means food which is capable of providing not only a nutritional effect, but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

Compositions of the present disclosure may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

Various aspects and embodiments of the present invention are defined by the following numbered clauses:

1. A method for promoting weight loss in a subject, comprising: administering to the subject a composition comprising from about 2 mg to about 800 mg of paraxanthine.
2. The method of clause 1, wherein paraxanthine is present in the composition in amount from about 20 mg to about 600 mg.
3. The method of clause 2, wherein paraxanthine is present in the composition in amount from about 50 mg to about 400 mg.
4. The method of any of clauses 1-3, wherein weight loss is promoted through inducing thermogenesis in the subject.
5. The method of clause 4, wherein the composition further comprises one or more compounds selected from a list consisting of: caffeine, green tea, capsaicin, garcinia cambogia, yohimbine and bitter orange.
6. The method of any of clauses 1-3, wherein weight loss is promoted through suppression of appetite in the subject.
7. The method of clause 6, wherein administration of the composition to the subject suppresses appetite in the subject by at least about 30%.
8. The method of clause 6, wherein the composition further comprises one or more compounds selected from a list consisting of: fenugreek, glucomannan, Gymnema sylvestre, 5-HTP, Caralluma fimbriata, green tea extract, Conjugated linoleic acid, Garcinia cambogia, and Yerba mate.
9. The method of any of clauses 1-3, wherein weight loss is promoted through enhancing lipolysis in the subject.
10. The method of any of clauses 1-3, wherein weight loss is promoted through increasing resting energy expenditure in the subject.
11. The method of clause 10, wherein administration of the composition to the subject decreases the respiratory quotient in the subject by at least about 10%.
12. The method of clause 10, wherein resting energy expenditure in the subject is increased by at least about 15%.
13. The method of clause 9, wherein the wherein the composition further comprises one or more compounds selected from a list consisting of caffeine, green tea extract, L-carnitine, Garcinia cambogia (hydroxycitric acid), capsaicin, ginseng, taurine, silk peptides and octacosanol.
14. The method of any proceeding clause, further comprising restricting calorie intake of the subject.
15. The method of clause 14, wherein the amount of weight loss in the subject is greater than that for a subject with an equivalent calorie restriction that has not been provided the composition.
16. The method of clause 14, wherein the ratio of fat loss to muscle loss in the subject is greater than that for a subject with an equivalent calorie restriction that has not been provided the composition.
17. The method of clauses 1-16, wherein the subject is not administered caffeine.
18. A method for promoting fat loss in subject, comprising:
providing the subject with a composition comprising about from 2 mg to about 800 mg of paraxanthine.
19. The method of clause 18, wherein paraxanthine is present in the composition in amount from about 20 mg to about 600 mg.
20. The method of clause 19, wherein paraxanthine is present in the composition in amount from about 50 mg to about 400 mg.
21. The method of clauses 18-20, wherein the subject is not administered caffeine.
22. The method of any of clauses 18-20, wherein fat loss is promoted through inducing thermogenesis in the subject.
23. The method of clause 22, wherein the composition further comprises one or more compounds selected from the list consisting of: caffeine, green tea, capsaicin, garcinia cambogia, yohimbine and bitter orange.
24. The method of any of clauses 18-20, wherein fat loss is promoted through suppression of appetite in the subject.
25. The method of clause 24, wherein administration of the composition to the subject suppresses appetite in the subject by at least about 30%.
26. The method of any of clauses 18-20, wherein administration of the composition to the subject decreases the respiratory quotient in the subject by at least about 10%.
27. The method of clause 24, wherein the composition further comprises one or more compounds selected from the list consisting of: fenugreek, glucomannan, Gymnema sylvestre, 5-HTP, Caralluma fimbriata, green tea extract, Conjugated linoleic acid, Garcinia cambogia, and Yerba mate.
28. The method of any of clauses 18-20, wherein fat loss is promoted through enhancing lipolysis in the subject.
29. The method of clause 28, wherein the wherein the composition further comprises one or more compounds selected from the list consisting of caffeine, green tea extract, L-carnitine, Garcinia cambogia (hydroxycitric acid), capsaicin, ginseng, taurine, silk peptides and octacosanol.
30. The method of any proceeding clause, further comprising restricting calorie intake of the subject.
31. The method of clause 30, wherein the amount of fat loss in the subject is greater than that for a subject with an equivalent calorie restriction that has not been provided the composition.
32. The method of clause 30, wherein the ratio of fat loss to muscle loss in the is greater than that for a subject with an equivalent calorie restriction that has not been provided the composition.
33. The method of any preceding clause, wherein the paraxanthine is derived from a natural source.
34. The method of any preceding clause, wherein the paraxanthine is synthetic.
35. A method for suppressing appetite in a subject comprising: administering to the subject a composition comprising from about 2 mg to about 800 mg of paraxanthine.
36. The method of clause 35, wherein paraxanthine is present in the composition in amount from about 20 mg to about 600 mg.
37. The method of clause 36, wherein paraxanthine is present in the composition in amount from about 50 mg to about 400 mg.
38. The method of clause 35, wherein administration of the composition decreases appetite in the subject by at least about 30%.
39. A method for enhancing lipolysis in a subject comprising: administering to the subject a composition comprising from about 2 mg to about 800 mg of paraxanthine.
40. The method of clause 39, wherein paraxanthine is present in the composition in amount from about 20 mg to about 600 mg.
41. The method of clause 40, wherein paraxanthine is present in the composition in amount from about 50 mg to about 400 mg.
42. The method of any preceding clause wherein the subject is overweight.
43. The method of clause 42, wherein the subject is obese.
44. A nutritional supplement for promoting weight loss comprising about 2 mg to about 800 mg paraxanthine and a nutraceutically acceptable carrier thereof.
45. The nutritional supplement of clause 44, wherein paraxanthine is present in amount from about 20 mg to about 600 mg.
46. The nutritional supplement of clause 45, wherein paraxanthine is present in amount from 50 mg to about 400 mg paraxanthine.
47. The nutritional supplement of clause 44, wherein the nutritional supplement is a dietary supplement in the form of a powder or capsule.
48. The nutritional supplement of clause 44, wherein the nutritional supplement is a functional food in the form of a beverage, nutrition bar, yoghurt, or cereal.
49. The nutritional supplement of clauses 40-32, wherein the nutritional supplement promotes weight loss through enhancing thermogenesis in a subject.
50. The nutritional supplement of clauses 44-49, wherein the nutritional supplement promotes weight loss through appetite suppression in a subject.
51. The nutritional supplement of clauses 44-49, wherein the nutritional supplement promotes weight loss through increasing resting energy expenditure.
52. The nutritional supplement of clauses 44-51, wherein the nutritional supplement promotes weight loss through reducing body fat in a subject.
53. The nutritional supplement of clause 52, wherein body fat is reduced through thermogenesis.
54. The nutritional supplement of clause 53, wherein body fat is reduced through increases lipolysis.
55. A method for increasing swagger in a subject, comprising:
providing the subject with a composition comprising about 2 mg to about 800 mg of paraxanthine.
56. The method of clause 55, wherein paraxanthine is present in the composition in amount from about 20 mg to about 600 mg.
57. The method of clause 56, wherein paraxanthine is present in the composition in amount from about 50 mg to about 400 mg.
58. The method of any of clauses 55-57, wherein the composition further comprises one or more compounds selected from the list consisting of: omega-3 fatty acids, vitamin D, vitamin B, protein, selenium, fast digestive carbohydrates like sugar, vitamin K, calcium, vitamin A, ashwagandha (Withania somnifera), Acetylcholine, Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, curcuminoids, caffeine, creatine, and Solanum asperum.
59. The method of any preceding clause wherein the paraxanthine is derived from a natural source.
60. The method of any preceding clause wherein the paraxanthine is synthetic.
61. A nutritional supplement for enhancing swagger comprising from about 2 mg to about 800 mg paraxanthine and a nutraceutically acceptable carrier thereof.
62. The nutritional supplement of clause 61 wherein the paraxanthine is present in amount from about 20 mg to about 600 mg.
63. The nutritional supplement of clause 61 wherein the paraxanthine is present in amount from about 50 mg to about 400 mg.
64. The nutritional supplement of any of clauses 61-63, wherein the nutritional supplement is a dietary supplement.
65. The nutritional supplement of clause 64, wherein the dietary supplement is powder or a capsule.
66. The nutritional supplement any of clauses 61-63, wherein the nutritional supplement is a functional food.
67. The nutritional supplement of clause 66, wherein the functional food is a beverage, nutrition bar, yoghurt, or cereal.
68. The nutritional supplement any of clauses 61-63, further comprises one or more compounds selected from the list consisting of: omega-3 fatty acids, vitamin D, vitamin B, protein, selenium, fast digestive carbohydrates, vitamin K, calcium, vitamin A, ashwagandha (Withania somnifera), Acetylcholine, Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, curcuminoids, caffeine, creatine, and Solanum asperum.
69. The nutritional supplement of any of clauses 61-68, wherein the paraxanthine is derived from a natural source.
70. The nutritional supplement of any of clauses 61-68, wherein the paraxanthine is synthetic.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

### Appetite

### Methods

Upon determination of baseline appetite via visual analog scale (VAS), two participants were assigned in a randomized, double-blind, placebo-controlled, crossover fashion to ingest a non-energetic placebo (maltodextrin) or a 100 mg dose of paraxanthine (PX), 300 mg of PX, 200 mg of caffeine (CA), or 200 mg PX plus 200 mg of CA. All supplements were orally ingested with 8 fluid ounces of cold tap water. The order of administration for all interventions was randomized using a random allocation software to ensure randomization and to avoid order effects. After ingestion, participants completed all assessments in an identical fashion 30, 60, 90, 120, and 180 minutes after ingestion of their assigned supplement. All study visits took place between 0600-1000 hours. At least 72 hours of rest will take place between each condition.

### Results

### Subjects Characteristics

| Gender | Male | Female |
|---|---|---|
| Age (years) | 23 | 24 |
| Height (cm) | 175 | 168 |
| Body Mass Index (kg/m2) | 28.5 | 24.1 |
| Fat Mass (kg) | 18.8 | 14 |
| Fat-Free Mass (kg) | 67.4 | 14.6 |
| % Fat | 21.8 | 20.6 |
| Screening Body Mass (kg) | 87.2 | 68.1 |
| Screening Heart Rate | 56 | 61 |
| (beats/min) | | |
| Screening Systolic Blood | 117 | 109 |
| Pressure (mm Hg) | | |
| Screening Diastolic Blood | 76 | 64 |
| Pressure (mm Hg) | | |

100 mg PX and 300 mg PX reduced mean appetite by 38.5%, and 43.1% respectively, compared to placebo, and showed greater reduction in appetite compared to CA. 200 mg CA showed a very marginal reduction in appetite of 2.4% compared to placebo. The minimal effect of caffeine was significantly increased by adding 200 mg PX, as the 200 mg PX plus 200 mg CA ingestion showed a 17.4% reduction in appetite.

Appetite Visual Analog Scale

| | | | Male, | Female, | | |
|---|---|---|---|---|---|---|
| | Condition | Time | age 23 | age 24 | Average | |
| Appetite | Placebo | 30 | 81 | 65 | | |
| Visual | | 60 | 90 | 60 | | |
| Analog | | 90 | 100 | 71 | | |
| Scale | | 120 | 100 | 80 | | |
| | | 180 | 100 | 90 | | |
| | MEAN | | 94.2 | 73.2 | 83.7 | |
| | 100 mg PX | 30 | 29 | 70 | | |
| | | 60 | 27 | 65 | | |
| | | 90 | 25 | 60 | | |
| | | 120 | 29 | 85 | | |
| | | 180 | 35 | 90 | | |
| | MEAN | | 29.0 | 74.0 | 51.5 | |
| | Δ vs. PLA | | −65.2 | +0.8 | −32.2 | (−38.5%) |
| | 300 mg PX | 30 | 24 | 65 | | |
| | | 60 | 30 | 60 | | |
| | | 90 | 29 | 55 | | |
| | | 120 | 27 | 60 | | |
| | | 180 | 26 | 80 | | |
| | MEAN | | 27.2 | 64.0 | 45.6 | |
| | Δ vs. PLA | | −67.0 | −9.2 | −38.1 | (−43.1%) |
| | Δ vs. 100 mg PX | | −1.8 | −10.0 | −5.9 | (−11.5%) |
| | 200 mg CA | 30 | 70 | 50 | | |
| | | 60 | 100 | 60 | | |
| | | 90 | 100 | 61 | | |
| | | 120 | 100 | 65 | | |
| | | 180 | 100 | 70 | | |
| | MEAN | | 94.0 | 61.2 | 77.6 | |
| | Δ vs. PLA | | −0.2 | −12.0 | −6.1 | (−2.4%) |
| | Δ vs. 100 mg PX | | +65.0 | −12.8 | +52.2 | (+101.3%) |
| | Δ vs. 300 mg PX | | +66.8 | −2.8 | +32.0 | (+70.2%) |
| | 200 mg PX + | 30 | 68 | 61 | | |
| | 200 mg CA | 60 | 71 | 65 | | |
| | | 90 | 80 | 65 | | |
| | | 120 | 82 | 60 | | |
| | | 180 | 60 | 79 | | |
| | MEAN | | 72.2 | 66.0 | 69.1 | |
| | Δ vs. PLA | | −22.0 | −7.2 | −14.6 | (−17.4%) |
| | Δ vs. 100 mg PX | | +43.2 | −8.0 | +17.6 | (+34.2%) |
| | Δ vs. 300 mg PX | | +45.0 | +2.0 | +23.5 | (+51.5%) |
| | Δ vs. 200 mg CA | | −21.8 | +4.8 | −8.5 | (−11.0%) |

### Example 2

### Fat Burning: Respiratory Quotient

### Methods

Upon determination of baseline respiratory quotient (RQ=VCO₂/VO₂) two participants were assigned in a randomized, double-blind, placebo-controlled, crossover fashion to ingest a non-energetic placebo (maltodextrin) or a 100 mg paraxanthine (PX). All supplements were orally ingested with 8 fluid ounces of cold tap water. The order of administration for all interventions was randomized using a random allocation software to ensure randomization and to avoid order effects. After ingestion, participants completed all assessments in an identical fashion 30, 60, 90, 120, and 180 minutes after ingestion of their assigned supplement. All study visits took place between 0600-1000 hours. At least 72 hours of rest took place between each condition.

### Results

The lower the RQ, the more fat that is being oxidized for body energy needs. The closer to 1.0 the more carbohydrate is being burned for energy needs. If numbers are lower, more fat is being oxidized. PX reduced the respiratory quotient by more than 10% compared to placebo, increasing fat oxidation (“fat burning”).

### Respiratory Quotient

| | | | Male, | Female, | |
|---|---|---|---|---|---|
| | Condition | Time | age 23 | age 24 | Average |
| Respiratory | Placebo | 0 | 0.77 | 0.70 | |
| Quotient | | 30 | 0.72 | 0.92 | |
| | | 60 | 0.85 | 0.72 | |
| | | 90 | 0.94 | 0.76 | |
| | | 120 | 0.80 | 0.88 | |
| | | 180 | 0.81 | 0.69 | |
| | MEAN | | 0.824 | 0.794 | 0.809 |
| | Mean Δ from 0 | | +0.054 | +0.094 | |
| | 100 mg PX | 0 | 0.72 | 0.74 | |
| | | 30 | 0.68 | 0.81 | |
| | | 60 | 0.66 | 0.74 | |
| | | 90 | 0.72 | 0.74 | |
| | | 120 | 0.70 | 0.69 | |
| | | 180 | 0.77 | 0.70 | |
| | MEAN | | 0.71 | 0.74 | 0.725 |
| | Δ vs. PLA | | −0.11 (−13%) | −0.05 (−6.3%) | −0.083 (−10.3%) |
| | MEAN Δ from 0 | | −0.014 | −0.004 | |

### Example 3

### Weight Loss, Energy Expenditure (PX)

### Methods

Upon determination of baseline resting energy expenditure, two participants were assigned in a randomized, double-blind, placebo-controlled, crossover fashion to ingest a non-energetic placebo (maltodextrin), 100 mg paraxanthine (PX), 200 mg PX or 300 mg PX. All supplements were orally ingested with 8 fluid ounces of cold tap water. The order of administration for all interventions was randomized using a random allocation software to ensure randomization and to avoid order effects. After ingestion, participants completed all assessments in an identical fashion 30, 60, 90, 120, and 180 minutes after ingestion of their assigned supplement. All study visits took place between 0600-1000 hours. At least 72 hours of rest took place between each condition.

### Results

PX supplementation increased resting energy expenditure in a dose-dependent manner from 9.7 to 16.9%, 186 to 304 kcal/day.

### Resting Energy Expenditure

| | | | Male, | | Female, | | | |
|---|---|---|---|---|---|---|---|---|
| | Condition | Time | age 23 | | age 24 | | Average | |
| Resting | Placebo | 30 | 2,034 | | 1,450 | | | |
| Energy | | 60 | 1,919 | | 1,829 | | | |
| Expenditure | | 90 | 1,723 | | 1,765 | | | |
| | | 120 | 2,153 | | 1,485 | | | |
| | | 180 | 1,905 | | 1,674 | | | |
| | MEAN | | 1,947 | | 1,641 | | 1,794 | |
| | 100 mg PX | 30 | 1,943 | | 1,690 | | | |
| | | 60 | 1,862 | | 1,809 | | | |
| | | 90 | 1,921 | | 1,899 | | | |
| | | 120 | 1,990 | | 1,787 | | | |
| | | 180 | 2,129 | | 1,796 | | | |
| | MEAN | | 1,969 | | 1,796 | | 1,883 | |
| | Δ vs. PLA | | +22 | (+1%) | +155 | (+9%) | +89 | (+5%) |
| | 200 mg PX | 30 | 2,007 | | 1,703 | | | |
| | | 60 | 1,702 | | 1,782 | | | |
| | | 90 | 2,048 | | 1,864 | | | |
| | | 120 | 2,221 | | 1,872 | | | |
| | | 180 | 2,067 | | 1,853 | | | |
| | MEAN | | 2,009 | | 1,815 | | 1,912 | |
| | Δ vs. PLA | | +52 | (+2.7%) | +174 | (+5.7%) | +113 | (+6.3%) |
| | Δ vs. 100 mg PX | | +40 | (+2.0%) | +19 | (+10.6%) | +30 | (+1.6%) |
| | 300 mg PX | 30 | 2,267 | | 1,840 | | | |
| | | 60 | 2,354 | | 1,749 | | | |
| | | 90 | 2,312 | | 1,769 | | | |
| | | 120 | 2,436 | | 1,831 | | | |
| | | 180 | 2,402 | | 1,913 | | | |
| | MEAN | | 2,374 | | 1,822 | | 2,098 | |
| | Δ vs. PLA | | +427 | (+21.9%) | +181 | (+11.0%) | +304 | (+16.9%) |
| | Δ vs. 100 mg PX | | +405 | (+20.5%) | +26 | (+1.5%) | +216 | (+11.4%) |
| | Δ vs. 200 mg PX | | +365 | (+18.2%) | +7 | (+0.4%) | +186 | (+9.7%) |

### Example 4

### Weight Loss, Energy Expenditure (PX+CA)

### Methods

In an experiment identical with Example 3, two participants were assigned in a randomized, double-blind, placebo-controlled, crossover fashion to ingest 200 mg caffeine plus 200 mg placebo (maltodextrin), or 200 mg caffeine plus 200 mg of PX. All supplements were orally ingested with 8 fluid ounces of cold tap water.

### Results:

The coadministration of 200 mg PX with 200 mg CA increased resting energy expenditure by an average of 168 kcals/day (+9%) over CA (male subject+166 kcals/day, female subject +170 kcals/day).

### Example 5

### Swagger

### Methods

Upon determination of baseline swagger and confidence via visual analog scale (VAS), two participants were assigned in a randomized, double-blind, placebo-controlled, crossover fashion to ingest a non-energetic placebo (maltodextrin) or a 100 mg dose of paraxanthine (PX), or a 200 mg dose of caffeine (CA). All supplements were orally ingested with 8 fluid ounces of cold tap water. The order of administration for all interventions was randomized using a random allocation software to ensure randomization and to avoid order effects. After ingestion, participants completed all assessments in an identical fashion 30, 60, 90, 120, and 180 minutes after ingestion of their assigned supplement. All study visits took place between 0600-1000 hours. At least 72 hours of rest took place between each condition.

### Results

100 mg PX ingestion increased swagger by 21% over placebo, and by 26% over 200 mg caffeine ingestion. Both, 100 mg PX and 200 mg of CA failed to increase confidence.

### Swagger Visual Analog Scale

| | | | Male, | Female, | | |
|---|---|---|---|---|---|---|
| | Condition | Time | age 23 | age 24 | Average | |
| Swagger | Placebo | 30 | 61 | 41 | | |
| (Visual | | 60 | 81 | 56 | | |
| Analog | | 90 | 81 | 50 | | |
| Scale) | | 120 | 80 | 60 | | |
| | | 180 | 90 | 60 | | |
| | MEAN | | 78.6 | 53.4 | 66.0 | |
| | 100 mg PX | 30 | 91 | 71 | | |
| | | 60 | 80 | 71 | | |
| | | 90 | 89 | 80 | | |
| | | 120 | 81 | 75 | | |
| | | 180 | 90 | 70 | | |
| | MEAN | | 86.2 | 73.4 | 79.8 | |
| | Δ vs. PLA | | +7.6 | +20.0 | +13.8 | (+21%) |
| | 200 mg Caffeine | 30 | 64 | 65 | | |
| | | 60 | 56 | 62 | | |
| | | 90 | 51 | 62 | | |
| | | 120 | 61 | 59 | | |
| | | 180 | 44 | 69 | | |
| | MEAN | | 55.2 | 63.4 | 59.3 | |
| | Δ vs. PLA | | −23.4 | +10.0 | −6.7 | (−10%) |
| | Δ vs. 100 mg PX | | −31.0 | −10.0 | −20.5 | (−26%) |

Confidence Visual Analog Scale

| | | | Male, | Female, | |
|---|---|---|---|---|---|
| | Condition | Time | age 23 | age 24 | Average |
| Confidence | Placebo | 30 | 70 | 75 | |
| (Visual | | 60 | 79 | 70 | |
| Analog | | 90 | 79 | 65 | |
| Scale) | | 120 | 90 | 70 | |
| | | 180 | 89 | 75 | |
| | MEAN | | 81.4 | 71.0 | 76.2 |
| | 100 mg PX | 30 | 70 | 70 | |
| | | 60 | 80 | 70 | |
| | | 90 | 89 | 65 | |
| | | 120 | 81 | 75 | |
| | | 180 | 90 | 70 | |
| | MEAN | | 82.0 | 70.0 | 76.0 |
| | Δ vs. PLA | | +0.6 | −1.0 | −0.2 (0%) |
| | 200 mg Caffeine | 30 | 80 | 74 | |
| | | 60 | 91 | 76 | |
| | | 90 | 71 | 66 | |
| | | 120 | 80 | 70 | |
| | | 180 | 90 | 65 | |
| | MEAN | | 82.4 | 70.2 | 76.3 |
| | Δ vs. PLA | | +1 | −0.8 | +0.1 (0%) |
| | Δ vs. 100 mg PX | | +0.4 | +0.2 | +0.3 (0%) |

### Example 6

### Swagger

Male, age 51, consumed 100 mg of paraxanthine, reports significant increases in swagger at the workplace, and during team sports. Feeling an undeniable force of energy and boost of bold self-assured behavior.

Female age 44, plays volleyball in the sand 2× week, nurse, consumed 200 mg of paraxanthine and reports an increase in swagger, feelings of unshakeable confidence.

Male, age 46, works out 4-5× week, uses coffee or energy drinks prior to workouts and might use coffee, energy drink or diet soda in the afternoon. The male subject consumed 400 mg of paraxanthine and reports a significant boost in swagger.

Male, age 52, plays soccer twice a week, consumed 50 mg of paraxanthine daily during two weeks, and reports increase in swagger, on and off the pitch, resulting in increased confidence in his skills, scoring more goals.

Although the disclosure has been described with reference to preferred embodiments, persons skilled in the art will recognize that changes may be made in form and detail without departing from the spirit and scope of the disclosed apparatus, systems and methods.

What is claimed is:

1. A method for promoting weight loss in a subject, comprising:
administering to the subject a composition comprising from about 2 mg to about 800 mg of paraxanthine.

2. The method of claim 1, wherein paraxanthine is present in the composition in amount from about 20 mg to about 600 mg.

3. The method of claim 2, wherein paraxanthine is present in the composition in amount from about 50 mg to about 400 mg.

4. The method of claim 1, wherein weight loss is promoted through inducing thermogenesis in the subject.

5. The method of claim 4, wherein the composition further comprises one or more compounds selected from a list consisting of: caffeine, green tea, capsaicin, garcinia cambogia, yohimbine and bitter orange.

6. The method of claim 1, wherein weight loss is promoted through suppression of appetite in the subject and wherein administration of the composition to the subject suppresses appetite in the subject by at least about 30%.

7. The method of claim 1, wherein weight loss is promoted through enhancing lipolysis in the subject.

8. The method of claim 1, wherein administration of the composition to the subject decreases the respiratory quotient in the subject by at least about 10%.

9. The method of claim 1, wherein resting energy expenditure in the subject is increased by at least about 15%.

10. The method of claim 7, wherein the wherein the composition further comprises one or more compounds selected from a list consisting of caffeine, green tea extract, L-carnitine, Garcinia cambogia (hydroxycitric acid), capsaicin, ginseng, taurine, silk peptides, catechols, epigallocatechin gallate (EGCG), catechins, and proanthocyanidins and octacosanol.

11. The method of claim 1, further comprising restricting calorie intake of the subject and wherein the amount of weight loss in the subject is greater than that for a subject with an equivalent calorie restriction that has not been provided the composition and wherein the ratio of fat loss to muscle loss in the subject is greater than that for a subject with an equivalent calorie restriction that has not been provided the composition.

12. The method of claim 1, wherein the subject is not administered caffeine.

13. A method for suppressing appetite in a subject comprising: administering to the subject a composition comprising from about 2 mg to about 800 mg of paraxanthine.

14. The method of claim 13, wherein administration of the composition decreases appetite in the subject by at least about 30%.

15. The method of claim 13, wherein the subject is not administered caffeine.

16. A method for increasing swagger in a subject, comprising:
providing the subject with a composition comprising about 2 mg to about 800 mg of paraxanthine.

17. The method of claim 16, wherein paraxanthine is present in the composition in amount from about 20 mg to about 600 mg.

18. The method of claim 17, wherein paraxanthine is present in the composition in amount from about 50 mg to about 400 mg.

19. The method of claim 15, wherein the composition further comprises one or more compounds selected from the list consisting of: omega-3 fatty acids, vitamin D, vitamin B, protein, selenium, fast digestive carbohydrates like sugar, vitamin K, calcium, vitamin A, ashwagandha (Withania somnifera), Acetylcholine, Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna, L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, curcuminoids, caffeine, creatine, and Solanum asperum.

20. The method of claim 15, wherein administration of the composition to the subject increases swagger in the subject by at least about 20%.

21. A method for promoting fat loss in subject, comprising:
providing the subject with a composition comprising about from 2 mg to about 800 mg of paraxanthine.

22. The method of claim 21, wherein paraxanthine is present in the composition in amount from about 20 mg to about 600 mg.

23. The method of claim 22, wherein paraxanthine is present in the composition in amount from about 50 mg to about 400 mg.

24. The method of claim 21, wherein the subject is not administered caffeine.

25. The method of claim 21, wherein fat loss is promoted through inducing thermogenesis in the subject.

26. The method of claim 25, wherein the composition further comprises one or more compounds selected from the list consisting of: caffeine, green tea, capsaicin, garcinia cambogia, yohimbine, catechols, EGCG, catechins, and proanthocyanidins and octacosanol and bitter orange.

27. The method of claim 21, wherein fat loss is promoted through suppression of appetite in the subject.

28. The method of claim 27, wherein administration of the composition to the subject suppresses appetite in the subject by at least about 30%.

29. The method of claim 21, wherein administration of the composition to the subject decreases the respiratory quotient in the subject by at least about 10%.

30. The method of claim 27, wherein the composition further comprises one or more compounds selected from the list consisting of: fenugreek, glucomannan, Gymnema sylvestre, 5-HTP, Caralluma fimbriata, green tea extract, conjugated linoleic acid, Garcinia cambogia, and Yerba mate.

31. The method of claim 21, wherein fat loss is promoted through enhancing lipolysis in the subject.

32. The method of claim 31, wherein the wherein the composition further comprises one or more compounds selected from the list consisting of caffeine, green tea extract, L-carnitine, Garcinia cambogia (hydroxycitric acid), capsaicin, ginseng, taurine, silk peptides and octacosanol.

33. The method of claim 21, further comprising restricting calorie intake of the subject.

34. The method of claim 33, wherein the amount of fat loss in the subject is greater than that for a subject with an equivalent calorie restriction that has not been provided the composition.

35. The method of claim 33, wherein the ratio of fat loss to muscle loss in the is greater than that for a subject with an equivalent calorie restriction that has not been provided the composition.
